(19) United States
(12) Patent Application Publication
(10) Pub. No.: US 2021/0299075 A1
(43) Pub. Date: Sep. 30, 2021

(54) Use of a virucidal preparation in the nasal cavity for prevention of transmission or contraction of viral illnesses, or to shorten the duration of, or lessen the severity of viral illnesses.

(71) Applicant: Kent Christopher New, Ponte Vedra Beach, FL (US)

(72) Inventor: Kent Christopher New, Ponte Vedra Beach, FL (US)

(21) Appl. No.: 17/214,900

(22) Filed: Mar. 28, 2021

Related U.S. Application Data
(60) Provisional application No. 63/003,162, filed on Mar. 31, 2020.

(51) Int. Cl. A61K 31/194; A61K 9/00; A61K 31/19; A61K 31/20; A61K 47/20; A61P 31/14

(52) U.S. Cl. CPC A61K 31/194; A61K 9/0043; A61K 31/19; A61M 31/00; A61K 47/20; A61P 31/14; A61K 31/20

(57) ABSTRACT

A method of reducing or inhibiting the contraction or communication of viral upper respiratory illnesses is proposed. Application of a virucidal preparation containing one or more organic acids (e.g. malic acid) with one or more surfactants (e.g. sodium C₁₄-C₁₆ olefin sulfonate) in a carrier agent (e.g. propanediol) to the nasal cavity of well individuals will reduce the chance that they contract a viral upper respiratory illnesses including coronavirus and influenza. Application of said preparation to the nasal cavity of individuals with a VURI will reduce the chance they transmit the illness by reducing self-contamination of their hands when touching their nose. The preparation is most effective when applied with a clean finger or applicator two or three times daily.

### CROSS-REFERENCE TO RELATED APPLICATIONS

Not Applicable

### STATEMENT REGARDING FEDERALLY SPONSORED RESEARCH OR DEVELOPMENT

No federally sponsored research was used in the development of this invention.

### REFERENCE TO SEQUENCE LISTING, A TABLE, OR A COMPACT DISC APPENDIX

Not applicable.

### BACKGROUND OF THE INVENTION

### 1. Field of the Invention

Embodiments of this disclosure relate to virucidal compositions for the prevention of the spread of viral illnesses, and/or to shorten the duration of and/or severity of viral illnesses such as viral upper respiratory infections (VURI, including the “common cold” and the “flu”). Preferred embodiments of this disclosure relate to the use of a virucidal preparation in the nasal cavity of uninfected individuals to reduce the chance that they contract a viral illness, especially after close contact with infected individuals or carriers. Embodiments of this disclosure also relate to preparations and the use thereof in the nasal cavity of infected individuals to reduce the severity and/or duration of viral illness.

The spread of viral illnesses such at VURI frequently occur through hand-to-hand transmission of virus followed by self-contamination with virus of the nasal, oral or conjunctival mucosa. Typically, an individual with a VURI will either directly or indirectly contaminate the hands of another individual who does not have the illness. The uninfected individual will thereby have viral particles on their hands. These viral particles initiate infection after the individual contaminates their own nasal, oral or conjunctival mucosa with the viral particles on their hands. In this way, although transmission occurs through the initial step of hand-to-hand transfer of viral particles, infection of new individuals typically occurs only if active viral particles gain access to the mucosa. Such access typically occurs via the face. More specifically, such access typically occurs via the mucous membranes of the face, such as the nasal, oral or conjunctival mucous membranes.

Of the many viral families that can cause VURI, coronavirus has proven to be particularly dangerous. The coronavirus strain known as SARS-CoV-1 caused an outbreak in 2002-04 which killed nearly 1000 people worldwide. SARS-CoV-1 was more lethal than typical coronavirus but not very contagious. Thus, the outbreak was relatively easily contained.

In the fall of 2019, a second coronavirus strain similar to SARS-CoV-1 known as SARS-CoV-2 was discovered in Wuhan, China. SARS-CoV-2 is able to cause severe respiratory illness similar to SARS-CoV-1 but is much more contagious and therefore lead to a worldwide pandemic infecting over 120 million and killing over 2.7 million people as of the time of this writing.

Due to the severity of the SARS-CoV-2 pandemic, extensive research into the pathogenesis of the virus has been conducted in 2020 and 2021. Numerous scientific publications have demonstrated the importance of the nasal cavity in the contraction of illness from SARS-CoV-2. Thus it is critical to develop methods of deactivating the virus in the nasal cavity to minimize or prevent disease following exposure to SARS-CoV-2.

Knowledge of the primary causative agents and the mechanism of transmission of viral illnesses have been in the public domain for over thirty years. Although considerable progress has been made in understanding the molecular biology of viruses responsible for these illnesses, little progress has been made in the prevention or treatment of viral illnesses. Efforts aimed at reducing transmission of viral illnesses have focused on developing products for use on the hands. These efforts fail largely due to two reasons: 1, the viruses responsible for VURI and other viral illnesses are difficult to inactivate; and 2, it is nearly impossible to keep one's hands free of viral contamination because they repeatedly come in contact with contaminated objects.

Certain embodiments of this disclosure aim to significantly reduce the spread of viral illnesses. Embodiments of this disclosure rely on the application of a broadly effective virucidal preparation to the nasal cavity, as numerous scientific studies have demonstrated the importance of the nasal cavity to the establishment of VURI.

### 2. Description of the Prior Art

The art in the prevention of transmission of VURI and other viral illnesses is extensive, but it has heretofore focused primarily on the inactivation of viral particles on the hands or face. Previous work lends insight into potential effective agents for use in this disclosure.

Hendley et al. investigated the antiviral activity of several compounds including iodine, ethyl alcohol, benzalkonium chloride (BAK), and hexachlorophene. These agents were known for their anti-bacterial activity, but all except iodine performed poorly as antiviral agents. Unfortunately iodine is too irritating to the skin to be frequently applied.

Poli et al. investigated the antiviral activity of several organic acids such as citric, malic, pyruvic, and succinic acids and found them to be effective against herpes simplex virus, rhabdovirus, and other enveloped viruses, but not effective against adenovirus, the only naked virus tested. Hayden, et al. reported that hand lotions containing 2% glutaric acid were more effective than placebo at inactivating certain serotypes of rhinovirus. Rhinoviruses are responsible for more colds than any other virus family, but not all colds. Therefore the use of organic acids alone as an antiviral agent will likely have some effectiveness at reducing the transmission of common cold viruses, but will not offer comprehensive protection against colds or other viral illnesses.

Snipes, et al. discovered that alcohols of certain chain lengths were effective at inactivating lipid-containing viruses. Unfortunately, the alcohols found most effective are extremely insoluble in aqueous media, making them somewhat difficult to employ in practical applications. However, further work with alcohols has lead to other useful antiviral inventions by Hendley, et al. and Konowalchuk, et al., described below.

Hendley, et al. (U.S. Pat. No. 6,034,133) disclose a virucidal hand lotion containing malic acid, citric acid, and a C1 to C6 alcohol which effectively inactivates rhinovirus contamination of treated hands. Their work also revealed that the use of lotions containing malic acid and citric acid without an alcohol would reduce, but not eliminate rhinovirus contamination of treated hands.

Konowalchuk, et al. (U.S. Pat. No. 7,045,548) disclose a method of inactivating viruses with a composition of a C1 to C3 monohydroxy alcohol or a C2 to C4 diol with a sufficient amount of an acid (either organic or inorganic) to adjust the pH of the composition to below 4.6. This is demonstrated effective in topically treating lesions caused by herpes simplex virus. No mention is made of the use of the composition in the nasal cavity to reduce transmission of viral illnesses. Further, this combination of ingredients has not been demonstrated to show efficacy against the viruses that cause VURI.

Brown-Skrobot, et al. (U.S. Pat. No. 4,975,217) disclose a method of inactivating viruses and bacteria through the application of a combination of an organic acid and an anionic surfactant, with or without an alcohol to the hands. This work describes the finding that a combination of an organic acid with an anionic surfactant has antiviral activity against both enveloped and naked viruses, thus is effective against a broad array of viruses able to cause VURI. The composition is described as a virucidal lotion for the hands.

Hossain, et al. (U.S. Pat. No. 4,897,304) disclose the use of a combination of organic acids and a surfactant in tissue paper to reduce the spread of viruses which cause VURI. Hossain discloses using tissue paper to wipe the nose to reduce the transmission of viral illness, but no mention is made of applying a preparation of the active agents to the nasal cavity. Further, Hayden, et al. reported that the use of tissue paper can interrupt the transmission of viruses whether treated with the antiviral composition or not. Hence no distinct advantage in reducing transmission of viruses which cause VURI is obtained by this product.

In summary, prior art contains several examples of various antiviral compositions for use on the hands or face to reduce transmission of viruses that cause VURI. No prior disclosure describes the use of an antiviral composition consisting of an organic acid plus a surfactant in the nasal cavity of healthy individuals to prevent transmission or contraction of viral illnesses.

### BRIEF SUMMARY OF THE INVENTION

Embodiments of this disclosure include virucidal preparations for and methods of:
reducing or inhibiting transmission of a viral illness to a user;
reducing or inhibiting transmission of a viral illness from a user;
and, reducing or inhibiting contraction of a viral illness.
   Reducing, as used in this disclosure, means decreasing the likelihood of something occurring. Inhibiting, as used in this disclosure, means stopping something from occurring.

As opposed to the plethora of products which attempt to keep hands free of viral contaminant, the goal of embodiments of this disclosure aims at inactivating virus in the nasal cavity. For example, embodiments of this disclosure include application of a gel, cream, ointment, or spray to deliver the antiviral ingredients to the nasal cavity.

Nasal cavity, as used in this disclosure, refers to the skin and the mucus membranes inside the nose.

Since contraction of VURI and other viral illnesses frequently occurs following contamination of the nasal mucosa with virus, preferred embodiments of this disclosure are effective at reducing contraction and/or transmission of a variety of viral illnesses. As preferred ingredients of preparations of this disclosure have broad anti-viral activity, use of embodiments of antiviral preparations of this disclosure have potential effectiveness against many antiviral illnesses including the common cold, influenza, herpes simplex virus (HSV) cold sores, viral gastroenteritis, roseola, viral conjunctivitis, and viral rhinosinusitis. Preferred embodiments of antiviral preparations of this disclosure are effective against viral upper respiratory infections (VURI). A VURI, as used in this disclosure includes, but is not necessarily limited to, infection with rhinovirus, adenovirus, influenza virus (e.g., Human A & B, Avian H9N2, and Swine H1N1), Human Coronavirus, Parainfluenza, Picornavirus, and Respiratory Syncytial Virus.

Embodiments of the virucidal preparation may contain a mixture of organic acids, with or without a surfactant in a carrier appropriate for delivery of the antiviral composition to the nasal cavity. The virucidal preparation may be used in well individuals to reduce the chance that they will contract a viral illness. The preparation may also be used by individuals with a viral illness to reduce the chance they transmit the viral illness.

Appropriate embodiments of this disclosure are described herein; however the description of these embodiments is not intended to limit the scope of the invention. For application to the nasal cavity, the antiviral composition may be prepared in appropriate carrier agents such as petrolatum, glycerin, mineral oil, dipropylene glycol, eucalyptus oil, dissolving creams, and xanthan gum. Appropriate pH for effective anti-viral preparations may be between 4 and 8, usually less than 5.

This disclosure also includes the development of appropriate applicators to assist in delivering the anti-viral preparation to the nasal cavity. Preferred embodiments include a small tube with a dome or beveled tip for application to the nasal cavity, or a cotton-tip swab for application deeper in the nasal cavity. The tube tip can have a single, centrally located hole or multiple peripherally located holes for extrusion of the ointment. This disclosure also includes kits containing both an applicator of the types discussed above and an anti-viral preparation.

### BRIEF DESCRIPTION OF THE SEVERAL VIEWS OF THE DRAWING

The disclosure will be described in greater detail in the following by way of example only with reference to various non-limiting embodiments as depicted in the annexed figures.

FIGS. 1a-b show dilution testing results for malic acid and sodium C₁₄-C₁₆ olefin sulfonate (Bio-Terge®). FIG. 1a demonstrates in vitro testing results for listed dilutions of test agent exposed to Rhinovirus Type 14 for 60 seconds. FIG. 1b demonstrates in vitro testing results for listed dilutions of test agent exposed to Rhinovirus Type 16 for 60 seconds. The table lists input titer, output titer and calculated log reduction in viral titer for each dilution.

FIGS. 2a-d show in vitro viral testing results for malic acid and sodium C₁₄-C₁₆ olefin sulfonate at two different concentrations (2a and b represent 0.4%/0.1%, 2c and d represent 2%/1%). Both test agents were investigated for antiviral activity against a panel of 24 viruses as listed. Exposure time of test agent to virus was 60 seconds.

FIGS. 3a-b show in vitro viral testing results for malic acid and sodium C₁₄-C₁₆ olefin sulfonate at two different concentrations (3a represents 0.4%/0.1%, 3b represents 2%/1%). Both test agents were investigated for antiviral activity against three different strains of human coronavirus. Exposure time of test agent to virus was 60 seconds.

### DETAILED DESCRIPTION OF THE INVENTION

Embodiments of this disclosure describe a powerful combination of agents which has the relatively uncommon property of being safe and tolerable on the skin and mucus membranes as well as possessing broad highly efficacious antiviral activity. This allows using the antiviral preparation in the nasal cavity to reduce the chance of contraction of a viral upper respiratory infection caused by a wide range of viral agents.

Embodiments of this disclosure described may be used in well individuals to reduce the chance that they will contract a viral illness. Alternatively, embodiments may be utilized in infected individuals to reduce the chance they spread the virus to others.

Embodiments of this disclosure are described below with preferred embodiments; however it should be understood that it is not intended to limit the invention to the embodiments described. The invention is intended to include all alternatives as may be included within the spirit and scope of the invention as defined by the appended claims.

Organic acids which are effective against viruses and are therefore appropriate for use in this disclosure include: valeric acid, lactic acid, glycolic acid, pelargonic acid, aspartic acid, malic acid, and citric acid. Appropriate embodiments of this disclosure include any combination of one or more of these organic acids in addition to a surfactant. One embodiment is malic acid (2% w/v) plus citric acid (2% w/v), with a surfactant in an appropriate carrier agent. Said ointment would be applied either directly using a finger, or indirectly using an applicator to the nasal cavity one or more times per day. Ideally the ointment is to be applied to the nasal cavity using a clean applicator several times daily.

Organic acids alone have been demonstrated to effectively inactivate only some rhinovirus serotypes, and are ineffective at inactivating adenovirus. However, the combination of organic acids and a surfactant, particularly alkyl sulfonates, has shown effectiveness at inactivating viruses from several families including: rhinovirus, adenovirus, influenza virus, coronavirus and parainfluenza virus. Members of these virus families are responsible for a majority of common colds and the flu in adults. Therefore, effective embodiment comprises, consists essentially of, or consists of a combination of one or more organic acids and one or more surfactants. A preparation or ointment that consists essentially of at least one organic acid, a surfactant and a carrier is limited to the at least one organic acid and surfactant as the only virucidal agents.

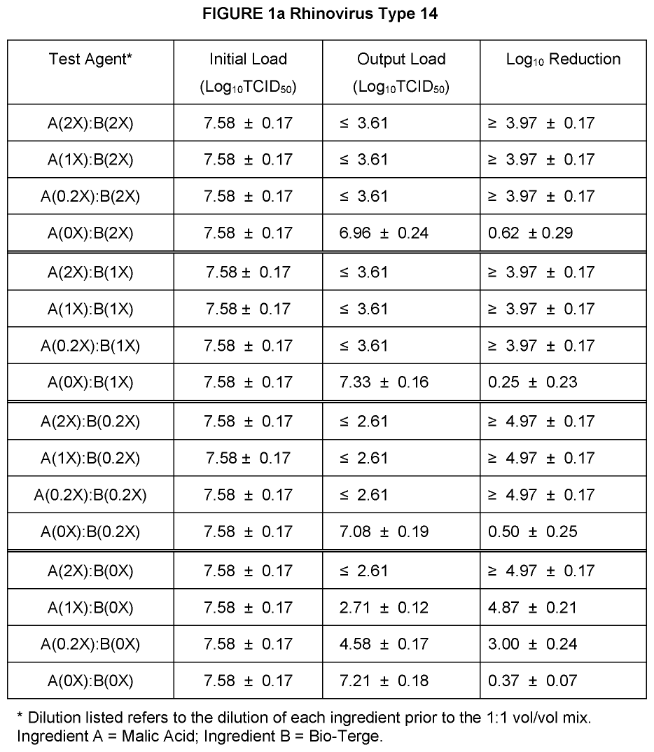

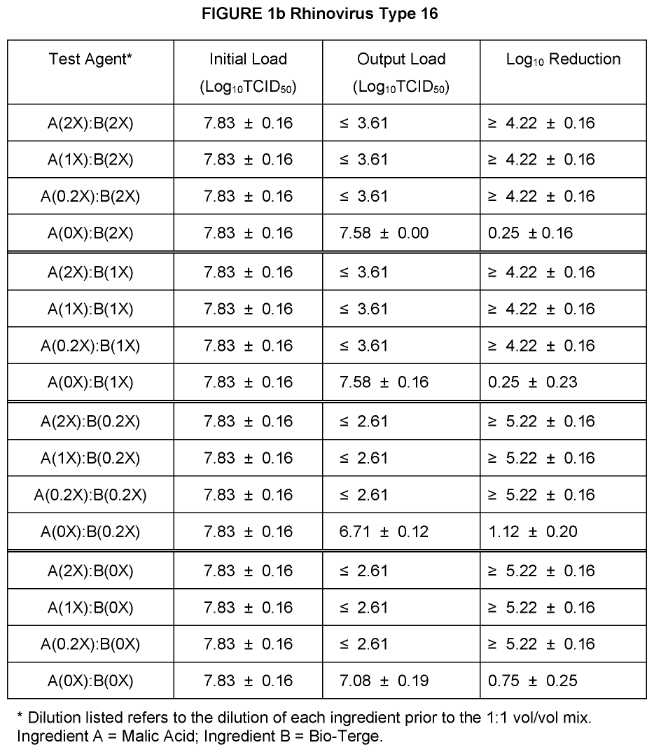

An independent microbiology laboratory has performed extensive in vitro testing of the combination of malic acid and sodium C₁₄-C₁₆ olefin sulfonate (Bio-Terge®) against several viruses. Clinical testing determined that malic acid 2% and Bio-Terge® 1% is very tolerable when applied to the nasal cavity up to three times daily. In vitro testing was then performed of serial dilutions of each ingredient starting with the maximum concentration of malic acid 2%/Bio-Terge® 1%. The dilution testing revealed that malic acid 0.4%/Bio-Terge® 0.1% resulted in greater than 10\hat{ }3 log reduction in virus titer of two serotypes of Rhinovirus; equal to the antiviral efficacy of the maximum concentration preparation tested. The dilution testing results are shown in FIGS. 1a and 1b. FIG. 1a shows dilution testing results obtained with malic acid and Bio-Terge®, the dilutions tested against Rhinovirus Type 14. FIG. 1b shows dilution testing results obtained with malic acid and Bio-Terge®, the dilutions tested against Rhinovirus Type 16.

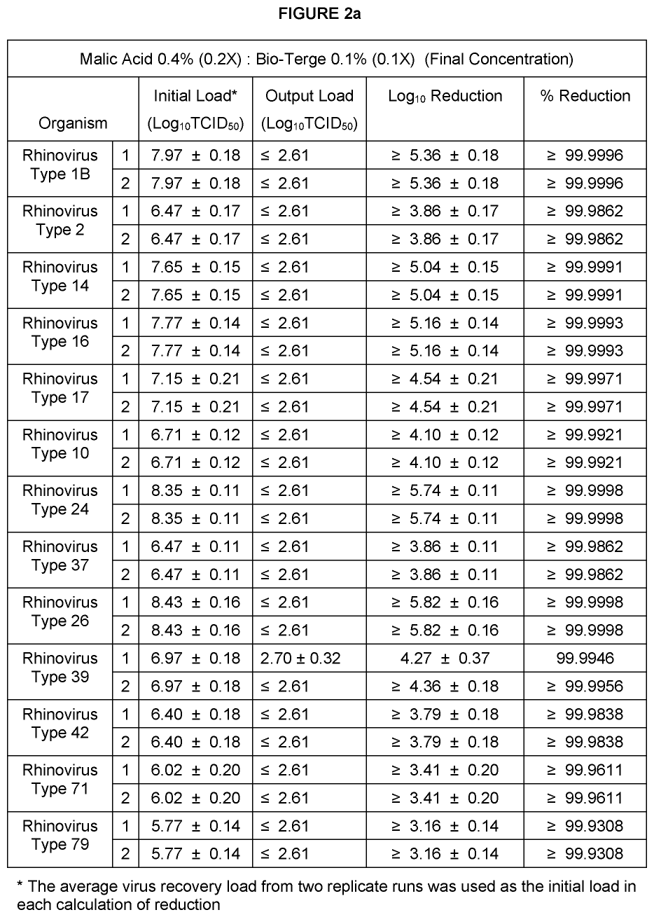

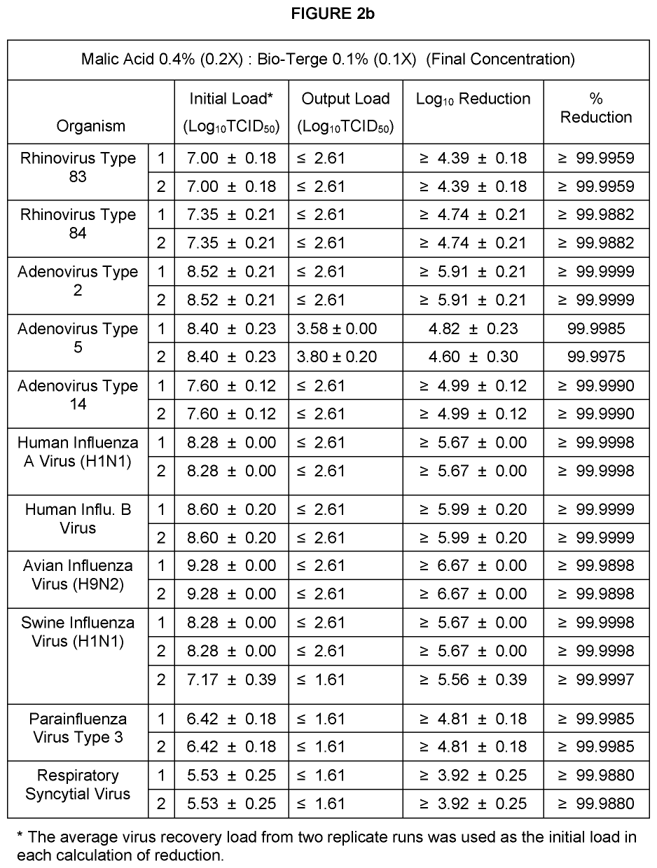

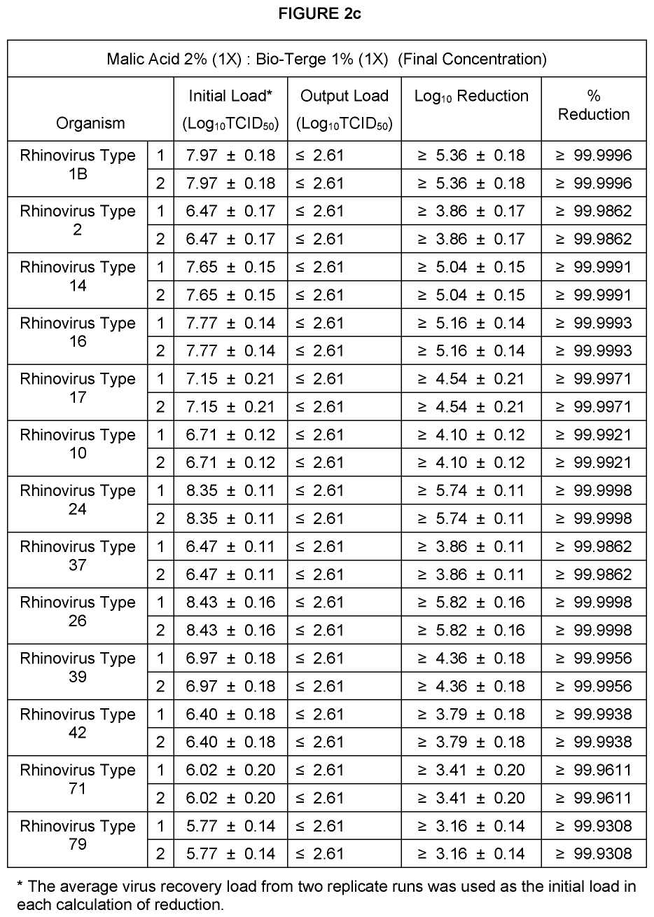

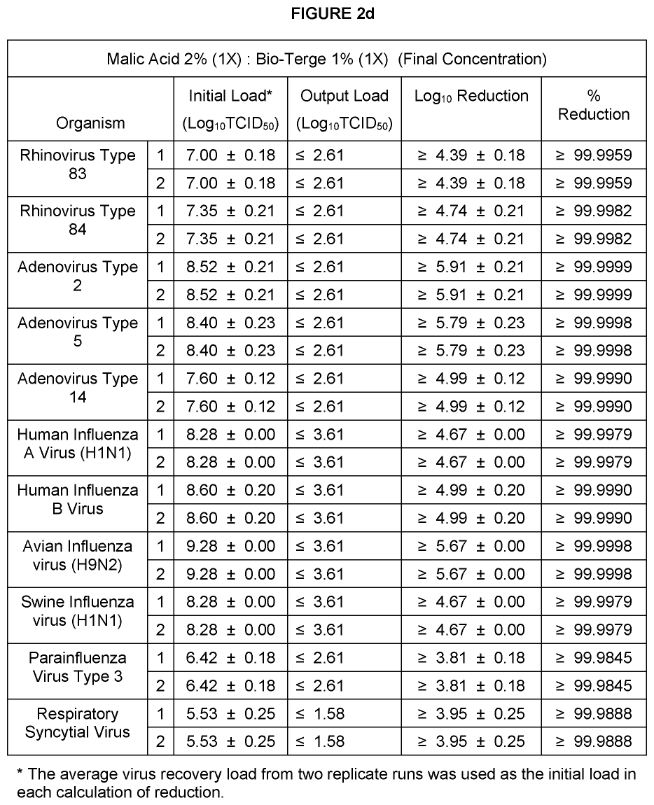

Following dilution testing, further in vitro testing was performed to evaluate the efficacy of two formulations (malic acid 2%/Bio-Terge® 1% and malic acid 0.4%/Bio-Terge® 0.1%) against a wide spectrum of viruses. Specifically, the formulations were tested against 15 serotypes of Rhinovirus, 3 serotypes of Adenovirus (2, 5, and 14), 4 strains of Influenza virus (Human A & B, Avian H9N2, and Swine H1N1), Parainfluenza virus type 3, and Respiratory Syncytial Virus. Both formulations demonstrated greater than 10\hat{ }3 log reduction in titer of all 24 viruses tested and greater than 10\hat{ }4 log reduction in 18 of 24 viruses (including all four influenza viruses) with a 60 second exposure time. The viral testing results are shown in FIGS. 2a-d.

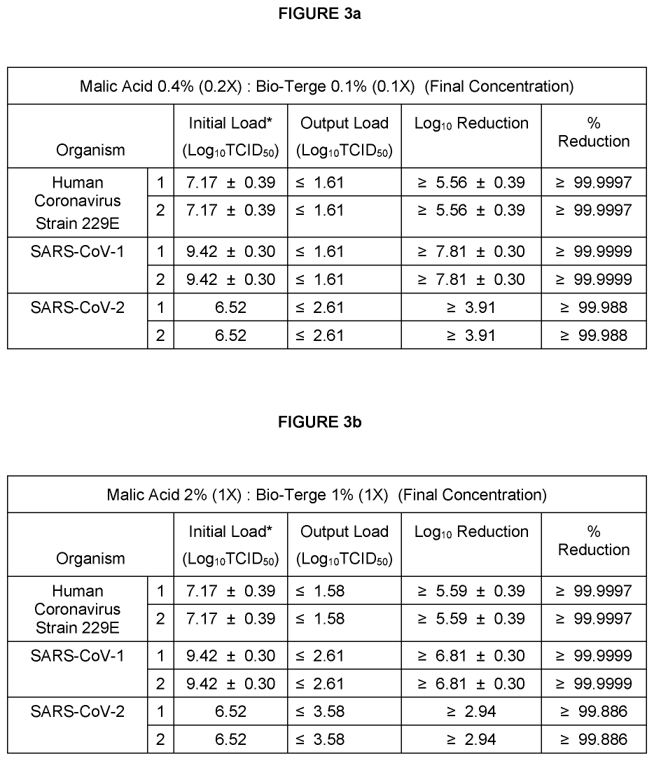

Further in vitro testing was performed to evaluate the efficacy of two formulations (malic acid 2%/Bio-Terge® 1% and malic acid 0.4%/Bio-Terge® 0.1%) against Human Coronavirus strain 229E, SARS-CoV-1, and SARS-CoV-2. Both formulations demonstrated greater than 10\hat{ }3 log reduction of strain 229E and SARS-CoV-1, and malic acid 0.4%/Bio-Terge®0.1% demonstrated 10\hat{ }3 log reduction of SARS-CoV-2. The higher concentration of acid and surfactant caused extensive cytotoxicity that limited the titration in testing against SARS-CoV-2. The cytotoxicity combined with the relatively low initial load of the virus resulted in the calculated log reduction greater than 10\hat{ }2.94. If a higher initial load was used it would be expected to show greater than 10\hat{ }3 log reduction as seen in other coronavirus testing. Exposure time was 60 seconds. The viral testing results are shown in FIGS. 3a-b.

A preparation or ointment may comprise, consist essentially of, or consist of up to 2% w/v malic acid and 1% w/v Bio-Terge®, preferably up to 1% w/v malic acid and 0.5% w/v Bio-Terge®, more preferably up to 0.4% w/v malic acid and 0.1% w/v Bio-Terge®.

Preliminary clinical research demonstrated safety and tolerability of the antiviral preparation in the nasal cavity. In addition, the preliminary clinical research has demonstrated effectiveness at reducing frequency of VURI illness.

Other surfactants with demonstrated effectiveness at inactivating viruses when combined with organic acids are suitable for this disclosure including: sodium dodecyl benzene sulfonate (BIOSOFT D-35X), sodium dodecyl sulfate (SDS), sodium lauryl sulfate (SLS), and sodium dioctyl sulfosuccinate (AEROSOL® OT). Appropriate concentration of the organic acid is 0.1-10% w/v, and of the surfactant is 0.1-10% v/v. Most effective and non-irritating embodiments include the organic acid at 0.1-5% w/v and the surfactant at 0.1-2% v/v.

Appropriate embodiments of this disclosure include any combination of one or more of the organic acids listed above with one of the surfactants listed in an appropriate carrier agent.

Appropriate viscosity for the nasal cavity +at 25° C. may be at least 10 P, or at least 25 P, or at least 50 P, or at least 200 P, or at least 1000 P.

In embodiments of the disclosure, the preparation is applied multiple times a day for prevention of contraction of influenza. Preferably, the application is at least once every 8 hours, at least one every 6 hours, at least once every 4 hours, or at least once every 2 hours.

In embodiments of the disclosure, the preparation is applied multiple times a day for prevention of contraction of coronavirus. Preferably, the application is at least once every 8 hours, at least one every 6 hours, at least once every 4 hours, or at least once every 2 hours.

While the present invention has been described with reference to various embodiments, it is to be understood that the invention is not limited to the disclosed embodiments. To the contrary, the invention is intended to cover various modifications and equivalent arrangements included within the spirit and scope of the appended claims.

1. A method of reducing the contraction or transmission of viral illnesses, said method comprising:
applying a virucidal preparation to the nasal cavity;
the preparation comprising one or more organic acids, one or more surfactants, and one or more carrier agents.

2. The method of claim 1, wherein the preparation is applied to the nasal cavity one to multiple times a day as a gel, ointment, cream, or spray.

3. The method of claim 1, wherein said organic acid is selected from the group consisting of valeric acid, lactic acid, glycolic acid, pelargonic acid, aspartic acid, malic acid, and citric acid.

4. The method of claim 1, wherein said surfactant is selected from the group consisting of sodium dodecyl sulfate, sodium lauryl sulfate, sodium dodecyl benzene sulfonate, sodium C₁₄-C₁₆ olefin sulfonate, and sodium dioctyl sulfosuccinate.

5. The method of claim 1, wherein said carrier agent is selected from the group consisting of petrolatum, glycerin, mineral oil, dipropylene glycol, eucalyptus oil and xanthan gum.

6. The method of claim 1, wherein the virucidal ointment comprises malic acid at a concentration of up to 2% w/v and sodium C₁₄-C₁₆ olefin sulfonate at a concentration of up to 1% v/v in a carrier agent.

7. The method of claim 1, wherein the virucidal ointment comprises malic acid at a concentration of up to 0.4% w/v and sodium C₁₄-C₁₆ olefin sulfonate at a concentration of up to 0.1% v/v in a carrier agent.

8. The method of claim 1, wherein the virucidal ointment contains one or more organic acids at a concentration between 0.1% and 10% w/v and a surfactant at a concentration between 0.1% and 10% v/v in a carrier agent.

9. A kit comprising:
an applicator; and
a virucidal preparation comprising one or more organic acids, one or more surfactants, and one or more carrier agents,
the applicator adapted for application of the virucidal preparation to the nasal cavity.
